(19) United States
(12) Patent Application Publication
(10) Pub. No.: US 2021/0077003 A1
(43) Pub. Date: Mar. 18, 2021

(54) SAMPLE COLLECTION DEVICE

(71) Applicant: SHIMADZU CORPORATION, Kyoto (JP)

(72) Inventor: Ippei TAKEUCHI, Kyoto (JP)

(21) Appl. No.: 16/971,833

(22) PCT Filed: Jan. 29, 2019
(86) PCT No.: PCT/JP2019/002865

§ 371 Date: Aug. 21, 2020

(30) Foreign Application Priority Data
Mar. 5, 2018 (JP) 2018-038159

(51) Int. Cl. A61B 5/15; A61M 1/36

(52) U.S. Cl. CPC A61B 5/150992; A61M 1/3672; A61B 5/150755

(57) ABSTRACT

A sample collection device is a sample collection device including a sample inlet and an internal flow passage for holding a sample sucked from the sample inlet. An anticoagulant for preventing coagulation of the sample is fixed to an inner surface of the inner flow passage of the sample collection device, and density of the anticoagulant fixed on the inner surface of an end portion near the sample inlet of the inner flow passage is higher than density of the anticoagulant fixed on the inner surface of an end portion on an opposite side to the sample inlet of the internal flow passage.

### TECHNICAL FIELD

The present invention relates to a sample collection device for collecting a small amount of blood in a flow passage and performing processing such as centrifugal separation.

### BACKGROUND ART

A sample collection device for collecting a small amount of sample has been proposed and implemented (see Patent Document 1). The sample collection device has a sample inlet for sucking a sample in an end portion, and a fine flow passage for holding a sample sucked from the sample inlet is provided inside. The flow passage is provided with thinness in such a way that a capillary force acts on a sample, and a sample can be taken into the flow passage simply by immersing the sample inlet in the sample.

When a blood sample is collected by the sample collection device, it is preferable to fix an anticoagulant on a surface of the flow passage of the sample collection device. By collecting a blood sample in the flow passage in which an anticoagulant is fixed, the anticoagulant can be dissolved in the blood sample collected by the sample collection device, and coagulation of the blood sample can be prevented.

### PRIOR ART DOCUMENTS

### Patent Documents

Patent Document 1: WO2016/009720A1

### SUMMARY OF THE INVENTION

### Problems to be Solved by the Invention

As a method of fixing an anticoagulant in a flow passage of a sample collection device, there is a method of filling the flow passage with a solution containing the anticoagulant and drying the solution, so that the anticoagulant is adsorbed on a flow passage surface. In this manner, the anticoagulant is uniformly fixed from one end side to the other end side communicating with the sample inlet in the flow passage of the sample collection device.

However, the anticoagulant fixed in the flow passage dissolves in blood at the same time as coming into contact with a blood sample taken in from the sample inlet depending on the solubility of the anticoagulant. Accordingly, there may be a situation in which the concentration of the anticoagulant is distributed so as to increase from one end side of the flow passage to the other end side, and, on one end side of the flow passage near the sample inlet, the anticoagulant is insufficient and coagulation of the blood sample cannot be restricted. To prevent such a situation, it is conceivable to fix an anticoagulant with a high concentration throughout the entire flow passage. However, in such a manner, the concentration of the anticoagulant becomes too high on the other end side of the flow passage, which may adversely affect the analysis of a blood sample.

In view of the above, an object of the present invention is to make it possible to dissolve an appropriate amount of an anticoagulant in an entire sample collected in a flow passage of a sample collection device.

### Solutions to the Problems

A sample collection device according to the present invention is a sample collection device including a sample inlet, and

an internal flow passage for holding a sample sucked from the sample inlet. An anticoagulant for preventing coagulation of the sample is fixed to an inner surface of the inner flow passage, and density of the anticoagulant fixed on the inner surface of an end portion near the sample inlet of the inner flow passage is higher than density of the anticoagulant fixed on an inner surface of an end portion on an opposite side to the sample inlet of the internal flow passage.

Density of the anticoagulant on the inner surface of the internal flow passage may be distributed so as to decrease in multiple steps from the end portion near the sample inlet to the end portion on an opposite side to the sample inlet.

### Effects of the Invention

According to the sample collection device of the present invention, an anticoagulant for preventing coagulation of a sample is fixed to an inner surface of an internal flow passage, and density of the anticoagulant is higher on an inner surface of a one end side portion of the internal flow passage than on an inner surface of the other end side portion. Accordingly, a difference in anticoagulant concentration in a sample generated between one end side and the other end side of the internal flow passage can be reduced as compared to a case where the anticoagulant is uniformly fixed from one end to the other end of the internal flow passage. This makes it possible to dissolve the appropriate amount of an anticoagulant in the entire sample collected in the internal flow passage.

### BRIEF DESCRIPTION OF THE DRAWINGS

FIG. 1 is a plan view showing an embodiment of a sample collection device.

FIG. 2 is a front view of the sample collection device.

FIG. 3 is a plan view showing an example of concentration distribution of an anticoagulant in an internal flow passage of the sample collection device.

FIG. 4 is data showing an analysis result of EDTA distribution in the internal flow passage by EPMA.

FIG. 5 is a plan view showing a measurement location of the sample collection device in verification of FIG. 4.

### EMBODIMENT OF THE INVENTION

An embodiment of a sample collection device will be described with reference to the accompanying drawings.

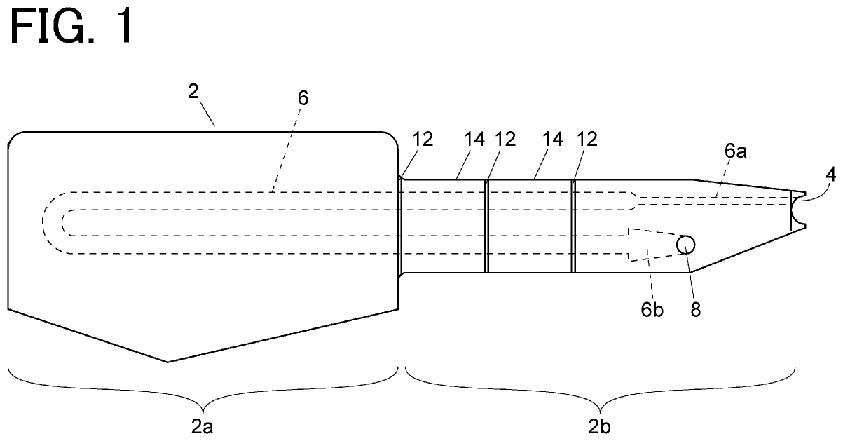

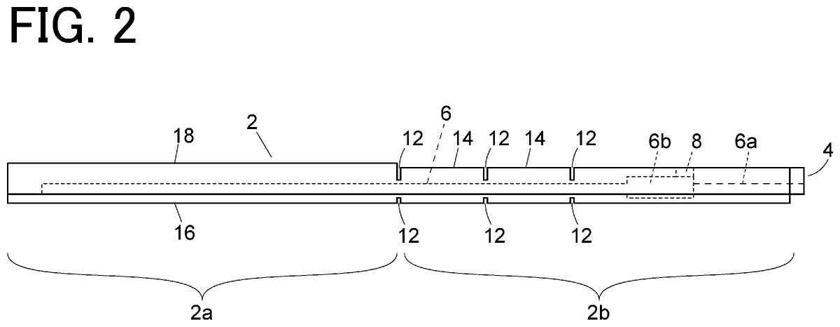

As shown in FIGS. 1 and 2, a sample collection device has a sample inlet 4 at a tip and an internal flow passage 6 having one end communicating with the sample inlet 4. The sample collection device 2 includes a base end portion 2a and a tip end portion 2b, and the width dimension of the tip end portion 2b is smaller than the width dimension of the base end portion 2a. The sample collection device 2 is configured by laminating a lower substrate 16 and an upper substrate 18 on top of each other, and the internal flow passage 6 is formed on a joint surface between the lower substrate 16 and an upper substrate 8.

The sample collection device 2 is made from, for example, a resin material. The resin material is not particularly limited, and, cycloolefin polymer (COP), polymethyl methacrylate resin (PMMA), polypropylene resin (PP), polycarbonate resin (PC), polyvinyl alcohol (PVA), and the like can be used.

The internal flow passage 6 has thinness that allows a sample to be sucked by the capillary phenomenon. The internal flow passage 6 has one end which communicates with the sample suction section 4 via a suction flow passage 6a, extends from there to the base end side of the sample collection device 2, and is folded back at the base end portion of the sample collection device 2 to extend to the tip end side. Liquid pool space 6b is provided at the other end of the internal flow passage 6, and the liquid pool space 6b is provided with an air hole 8 that serves as an air outlet when a sample is taken into the internal flow passage 6 by the capillary phenomenon. The air hole 8 is formed as a through hole penetrating the upper substrate 18.

The tip end portion 2b of the sample collection device 2 is provided with an extraction portion 14 that can be cut at a cutting groove 12. The extraction portion 14 is defined by two of the cutting grooves 12 which are parallel to each other. The cutting groove 12 is formed in a direction orthogonal to a longitudinal direction of the internal flow passage 6 and extends over the entire width of the tip end portion 2b.

After collecting a blood sample in the internal flow passage 6, the sample collection device 2 is placed in a centrifuge with its tip end side facing a rotation center side so that the blood sample can be centrifuged. A position where the extraction portion 14 is provided is set to a position where a plasma component or a serum component after separation comes when a sample collected in the internal flow passage 6 is subjected to centrifugal separation processing. In this manner, a plasma component or a serum component can be easily extracted only by dividing the extraction portion 14 from the sample collection device 2 after the centrifugal separation processing.

The liquid pool space 6b has, at least in its inlet portion, a cross-sectional area of size that does not allow liquid to be sucked by the capillary phenomenon. The liquid pool space 6b has an internal volume that is equal to or more than an internal volume of portion closer to the tip end side than the air hole 8 of the suction passage 6a. Since the liquid pool space 6b does not suck a sample by the capillary phenomenon, a sample sucked from the sample inlet 4 stops at an inlet portion of the liquid pool space 6b without reaching the position of the air hole 6. When centrifugal separation is performed in this state, an excess sample due to an equilibrium state of the sample is stored in the liquid pool space 6b, and the excess sample is prevented from overflowing from the internal flow passage 6 and coming out from the air hole 8.

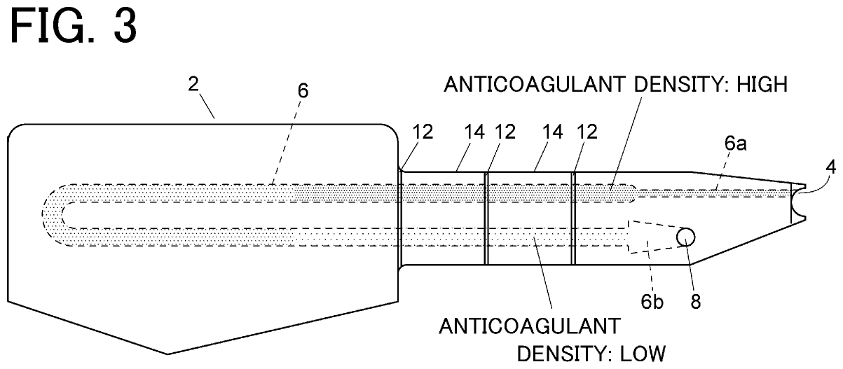

An anticoagulant is fixed on a surface inside the internal flow passage 6 of the sample collection device 2. The anticoagulant is for preventing coagulation of a blood sample sucked into the internal flow passage 6 by dissolving in the blood sample. As shown in FIG. 3, the density of the anticoagulant fixed on the surface in the internal flow passage 6 (an anticoagulant amount per unit area) is distributed so as to be highest on one end side of the internal flow passage 6 close to the sample inlet 4 and lowest on the other end side of the internal flow passage 6 far from the sample inlet 4. In FIG. 3, anticoagulant density distribution in three stages is formed in the internal flow passage 6. However, the present invention is not limited to this, and density distribution of two stages or four stages or more may be formed.

As described above, the anticoagulant density is highest on one end side of the internal flow passage 6 close to the sample inlet 4, so that, when a blood sample is collected in the internal flow passage 6, a situation in which a sufficient amount of the anticoagulant is not supplied to the blood sample taken into one end side of the internal flow passage 6 and the effect of preventing coagulation of the blood sample cannot be sufficiently obtained is prevented. Further, since the anticoagulant density is lowest on the other end side of the internal flow passage 6 far from the sample inlet 4, a situation in which the anticoagulant concentration in the blood sample taken into the other end side of the internal flow passage 6 becomes too high is prevented.

As the anticoagulant, ethylenediaminetetraacetic acid (EDTA), heparin, sodium citrate, double oxalate, and the like can be used according to the purpose of inspection.

As a method of forming the distribution of the anticoagulant density as described above in the internal flow passage 6, there is a method, in which a plurality of types of solutions, which are solutions (solvent or, for example, water) containing an anticoagulant and have different anticoagulant concentrations, are prepared, and introduced into the internal flow passage 6 from the sample inlet 4 of the sample collection device 2 in order from a solution with a lower anticoagulant concentration, and then the solvent in the internal flow passage 6 is dried. At the time of introducing each solution into the internal flow passage 6, a required amount of each solution can be introduced into the internal flow passage 6 accurately by directly injecting the solution into the internal flow passage 6 from the sample inlet 4 using a measuring instrument such as a micropipette.

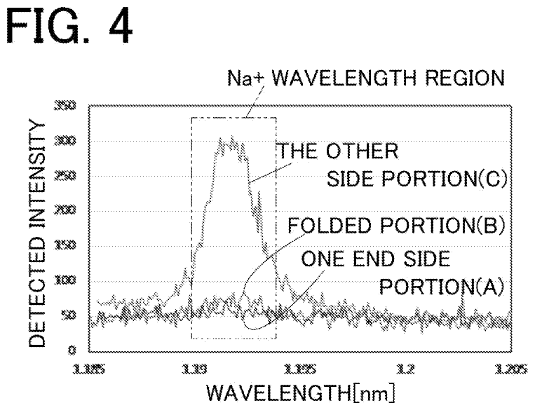

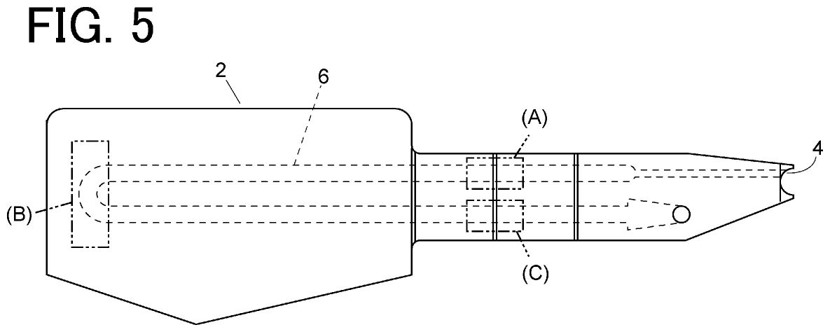

FIG. 4 shows data of verification of EDTA-2Na concentration distribution in the internal flow passage 6 that was carried out by using an electron beam microanalyzer (EPMA) after two types of solutions having different concentrations of EDTA-2Na are introduced into the internal flow passage 6 in order from a solution having a high concentration of EDTA-2Na and dried. In this verification, the Na-Ka line intensity was measured in (A) a one end side portion, (B) a folded portion, and (C) the other end side portion of the internal flow passage 6 shown by a two-dot chain line in FIG. 5.

A wavelength region of 1.19 nm-1.195 nm of Na⁺ in the verification data of FIG. 4 shows that the detected intensity is highest in (C) the other end side portion, while the detected intensity is lower in (A) the one end side portion and (B) the folded portion than the above. This indicates that existence densities of EDTA-2Na on the one end side and the other end side of the internal flow passage 6 are different. This has proved that it is possible to form the distribution of the anticoagulant density in the internal flow passage 6 by sequentially introducing a plurality of types of solutions having different anticoagulant concentrations into the internal flow passage 6 and drying the solutions.

### DESCRIPTION OF REFERENCE SIGNS

2: Sample collection device
2a: Base end portion
2b: Tip end portion
4: Sample inlet
6: Internal flow passage
6a: Sample suction flow passage
6b: Liquid pool space
8: Air hole
12: Cutting groove
14: Extraction portion
16: Lower substrate
18: Upper substrate

1. A sample collection device comprising:
a sample inlet; and
an internal flow passage for holding a sample sucked from the sample inlet, wherein
an anticoagulant for preventing coagulation of the sample is fixed to an inner surface of the inner flow passage, and density of the anticoagulant fixed on the inner surface of an end portion near the sample inlet of the inner flow passage is higher than density of the anticoagulant fixed on the inner surface of an end portion on an opposite side to the sample inlet of the internal flow passage.

2. The sample collection device according to claim 1, wherein density of the anticoagulant on an inner surface of the internal flow passage is distributed so as to decrease in multiple steps from the end portion near the sample inlet to the end portion on the opposite side to the sample inlet.
